(19) United States
(12) Patent Application Publication
(10) Pub. No.: US 2002/0197273 A1
(43) Pub. Date: Dec. 26, 2002

(54) Antitumor/immunocompetent supplementary healthy foods

(76) Inventor: Shozo Umezawa, Koshigaya-shi (JP)

Correspondence Address: ARMSTRONG,WESTERMAN & HATTORI, LLP, 1725 K STREET, NW., SUITE 1000, WASHINGTON, DC, 20006, US

(21) Appl. No.: 10/206,351

(22) Filed: Jul. 29, 2002

Related U.S. Application Data
(63) Continuation-in-part of application No. 09/960,397, filed on Sep. 24, 2001, now abandoned.
Application No. 09/960,397 is a continuation-in-part of application No. 09/691,184, filed on Oct. 19, 2000, now abandoned.

(30) Foreign Application Priority Data
Apr. 18, 2000 (JP) 2000-116134

(51) Int. Cl. A61K 35/84; A61K 35/78

(52) U.S. Cl. 424/195.15; 424/725; 426/18

(57) ABSTRACT

Healthy foods having both antitumor activity and activation effect for cell regeneration ability, comprising mycelia of mushrooms and one or plurality of fermented crop products, preferably, at a blending ratio of 30 to 50% by weight of mycelia and from 65 to 45% by weight of one or a plurality of fermented crop products in one embodiment, mycelia of mushrooms one or plurality of fermented crop products and calcium added thereto, preferably, at a blending ratio of 30 to 50% by weight of mycelia, from 65 to 45% by weight of fermented crop products and from 0.5 to 3% by weight of calcium in another embodiment, the fermented crop products being preferably prepared by fermenting and ripening vegetables, fruits and rood crops, and one or plurality of lytic enzymes being possibly added to the healthy foods.

### BACKGROUND OF THE INVENTION

1. Field of the Invention

The present invention concerns healthy foods for giving damages to neoplasms such as malignant tumors, as well as recovering and activating immunocompetence of living bodies.

2. Related Art Statement

It is considered that chemotherapy and radiotherapy against malignant tumors (hereinafter referred to as cancers) give damages not only to cancer cells but also to normal cells and lowers immunocompetence to result in poor progonostic state and frequent recidivation, so that various preventions and treatments of cancers by immunotherapy have been attempted.

As antitumor agent to be used for the immunological treatment, it have been known carcinostatic or healthy foods prepared by extracting fruit bodies of mushrooms (raw shiitake: Lentinus edodes) with hot water, and separating and purifying carcinostatic substances. In addition, it has been known that mycelia extracted from solid culture media of mushrooms (mainly comprising, for example, rice plant straws and cane lees) contain antitumor polysaccharide substances.

Antitumor ingredients of anticancer drugs extracted from fruit bodies and mycelia of mushrooms are polysaccharide substances mainly comprising β-glucanes such as β-1-3 glucanase and β-1-4 glucanase, but anticancer drugs extracted from fruit bodies are not so effective for inhibiting tumors of human bodies in view of the mass of polysaccharides. Therefore, although they exhibit high antitumor activity in experiments using mice, no expected carcinocidal activities can be obtained against cancers of human bodies. Then, though they are admitted as anticancer drugs combined use with other anticancer drugs is obliged at present. Further, even when they are used in combination, it is said that their effective antitumor activity is only less than ten and several percent.

On the other hand, the mass of polysaccharide substances contained in mycelia is 50 times in the concentration or 100 times in the amount relative to that of fruit bodies, and strong antitumor activity has been expected to some extent. However, although the mycelia have an antitumor activity by their high mass content of polysaccharide substances, no sufficient tumor inhibiting effect can be provided since their activity to activate peripheral aplastic regeneration cells is poor.

Accordingly, an object of the present invention is to provide healthy foods containing fermented crop products having both antitumor activity due to high mass content of polysaccharide substances and activation of cell regeneration ability due to fermented crop products.

### SUMMARY OF THE INVENTION

For attaining the object, the healthy foods of the present invention comprises mycelia of mushrooms and one or plural kinds of fermented crop products by using an ordinary stirring method.

Among fermented crop products, fermented crop products having antioxidant activity are particularly preferred. It is said the disorders of cells are caused by oxidation of cells, which transform to aplastic cells to result in cancers. However, it is considered that even if cells become aplastic, the regeneration ability recovers by reduction/oxidation of the cells unless they are completely damaged by cancer cells, so that the immunocompetence is enhanced to suppress growth of cancer cells in combination with the antitumor activity of the polysaccharides and further annihilate cancer cells. Namely, since cancer cells grow rapidly, the food composition of the invention should contain immediately effective massive cancerocidal substances, which can also activate immunocompetence of cells in the aplastic state.

The healthy foods of this invention is attained by the antitumor activity of polysaccharides contained by a great mass in mycelia of mushrooms and the immuno-activity of fermented crop products.

In this case, “anti-tumor activity” means an activity of suppressing generation of human tumors or an activity of reducing or annihilating generated tumors, and the “immunoactivity” means an activity of activating the immnocompetence of living cells.

The healthy foods of this invention suppress tumorigenesis and reduce or annihilate generated tumors when they are orally intaken as foods as they are, or orally intaken being added to other foods by the action of polysaccharides such as β-1-3-glucanase and β-1-4-glucanase contained by a great mass in mycelia of mushrooms, as well as give ingesta necessary for maintaining the life of cells while preventing oxidation of cells to activate the immunocompetence by the action of the fermented crop products. In addition, referring to the immunoactivating mechanism of the fermented crop products, it is considered that regenerating or recovering genes that control the regeneration ability of cells react with the fermented crop products to enhance the regeneration ability of the cells and activate the immunocompetence.

Namely, the healthy foods of this invention activate the immunocompetence of normal cells while damaging cancer cells, or give damages to cancer cells while activating immunocompetence of normal cells. The anti-tumor activity and the immunoactivating ability synergistically act on cells thereby capable of giving fatal damages to the cancer cells.

The mixing ratio of the mycelia and fermented crop products is from 30 to 50% by weight of mycelia of mushrooms and from 70 to 50% by weight of fermented crop products and, more preferably, 40% by weight of mycelia and 60% by weight of the fermented crop products.

In the healthy foods of this invention, calcium component may further be added to the mixture of mycelium and fermented crop products in order to further enhance the regeneration ability and the immunoactivity of living cells. The mixing ratio in this case is preferably from 30 to 50% by weight of the mycelia, from 65 to 35% by weight of the fermented crop products and from 0.5 to 3% by weight of the calcium component (as calcium basis) and, more preferably, 40% by weight of mycelia, 50% by weight of the fermented crop products and 1% by weight of calcium.

In the healthy foods of the present invention, lytic enzymes may be added to the mixture described above. By adding the lytic enzymes, the antitumor component and the cell activating component of the healthy foods of the present invention can be intaken and absorbed more easily to cancer cells and aplastic cells.

Preferred Embodiments of the Invention

The healthy foods according to the present invention are prepared by mixing one or plurality of fermented crop products in the mycelia of mushrooms as described above, and they also include those comprising such mixture as the main material and other materials added thereto.

Referring to the mushrooms to be used, shiitake (Lentinus edodes) is preferable in view of the availability of raw materials, and it has been found that edible mushrooms such as Pleurotus, Flammulina velutipes, Pholiota nameko and Basidiomycota such as Plypraceae and Coniolus versilor also have antitumor activity (carcinocidal activity), so that the mycelia of them may also be used.

The mycelia of Shiitake can be prepared by inoculating shiitake fungi in solid culture media for culturing shiitake (preferably, those prepared from materials comprising rice plant straws, sawdusts, bamboo grasses and bagasses as main materials and various ingesta such as defatted rice bran or the like added thereto), liquefying them at the timing of a full-ripe stage (culture period: about three months) of the mycelia in the culture media, and powderizing them in the activated state as they are. It will be apparent that they can be produced by other production processes.

The mycelia of mushrooms contained in the healthy foods according to the present invention are advantageously prepared as follows.

(1) inoculating Shiitake fungi to solid culture medium and culturing them for about four months,

(2) pulverizing the culture medium at an appropriate timing of growth of white mycelium on the surface of the medium, adding water thereto and warming them to about 60° C., thereby promoting liquefaction, that is, hydrolysis by the effect of enzyme groups and, simultaneously, squeezing the same as a slurrified liquid after confirming the completion of the enzymatic reaction of the mycelium,

(3) filtering and sterilizing the squeezed slurrified liquid and then concentrating the same at about 40° C., followed by powderization.

The fermented crop products (antioxidation substance) to be mixed with the mycelia, when taken into living bodies, prevent oxidation of cells, or give ingesta to living cells which have been rendered aplastic by oxidation thereby recovering and activating their immunocompetence.

The fermented crop products to be used in the healthy foods of this invention can be obtained by fermentation of starting materials comprising, as a main material, vegetables such as broccoli and lettuce, fruits such as apples and grapes and root crops such as carrots and beats, or mixtures thereof.

The fermented crop products contained in the healthy foods according to the present invention are advantageously prepared from the starting materials as follows:

(1) mixing the materials described above with yeast, water and maltose,

(2) keeping the mixture at a temperature of about 60 to about 80° C., preferably about 70° C., for about 4 to about 7 days, preferably, for about 5 days, so that aerobic fermentation by aerobic bacteria takes place,

(3) keeping the fermented intermediate products at a temperature of about 30 to about 50° C., preferably, about 40° C., for about 5 to about 10 days, preferably, for about 6 days, and aging them under sterilization at low temperature, and

(4) causing liquid extracts obtained by squeezing the products formed by the process described above to be adsorbed on wheat powder and then powderizing them by a grinder.

The fermented crop products obtained by the process described above are material having high energy, containing various kinds of enzymes such as oxidation preventive enzymes and nutritive ingredients for enhancing and activating immunocompetence of human cells.

In particular, when the mycelia of shiitake contains various lytic enzymes, they have an effect of making antitumor components and oxidation preventive substances of the healthy foods readily absorbable to cells. Optionally, enzymatic reactions by various lytic enzymes may be taken place before liquefication of collected hyphae in the production process of Shiitake mycelia, and various lytic enzymes such as protease, glycolase and lipase may be additionally bonded or mixed to the mycelia of Shiitake. The lytic enzyme is preferably a lytic enzyme containing antitumor polysaccharides.

An appropriate mixing ratio of mycelia and fermented crop products s is from 30 to 50% by weight of mycelia and from 70 to 50% by weight of fermented crop products and, preferably, 40% by weight of mycelia and 60% by weight of fermented crop products. Mixing ratio of the mycelia of less than 30% by weight is not preferred since polysaccharides such as 0-1-3 glucanase and β-1-4-glucanase having antitumor activity are insufficient. Further, mixing ratio of the fermented crop products of less than 50% by weight is not preferred since antioxidation ability of cells is lowered to result in disorders in activation of immunocompetence.

As another embodiment of the present invention, fermented crop products and calcium may be mixed with mycelia of mushrooms to enhance the recovery and regeneration ability and the immunocompetence of cells by the activating ability of calcium.

As the calcium component to be used in the healthy foods of this invention commercially available calcium preparations obtained by extracting from sea weeds such as kelp as the raw material can be used.

The mixing ratio in the example is from 30 to 50% by weight of the mycelia, from 65 to 45% by weight of the fermented crop products and from 0.5 to 3%0 by weight of calcium, and, more preferably, 40% by weight of the mycelia, 59% by weight of the fermented crop products and 1% by weight of calcium.

The healthy food according to the present invention in which calcium is added, for example, by 1% by weight to the mixture of the mycelia of mushrooms and the fermented crop products is a material having high energy, containing antitumor ingredients such as β-glucan of the mycelia of mushrooms, and oxidation preventive enzymes and various nutritive ingredients of the fermented crop products contained in admixture.

The healthy foods of this invention are orally intaken by 10 to 20 g per one day for an adult person as they are or being mixed with other foods.

### EXAMPLES

Mycelia of Shiitake

Shiitake fungi were re inoculated to solid medium for culturing shiitake, ripen by cultivating the hypae for 3 months, and subjected to liquefication to obtain 200 g of Shiitake mycelia powdered in an activated state as it was.

Fermented Crop Products

100 g of yeast, 500 cc of water and 100 g of maltose were added to a mixture of broccoli, lettuce, apples, carrots and beets in an amount of 1 kg in total and fermented and ripened. Liquid extracts obtained from the product were adsorbed to 200 g of wheat powder, dried and subsequently powdered by a grinder to obtain 200 g of powdery fermented crop products.

Calcium

Commercially available calcium preparation (powder) purified from kelp as the raw material was provided.

49 g of mycelia of shiitake, 50 g of fermented crop products and 1 g of calcium were charged into a stirrer, stirred and mixed at 60 rpm for 10 min to prepare 100 g of powdery healthy foods of this invention in the purified form. The result of the analysis for the ingredients of the healthy foods obtained as described above were as shown below. Result of Analysis for the Ingredients of Healthy Foods

| | |
|---|---|
| Moisture (air oven method) | 2.4 g/100 g |
| Protein | 7.1 g/100 g |
| Fat (Method with acid hydrolysis) | 1.5 g/100 g |
| Ash | 2.9 g/199 g |
| Carbohydrates | 86.1 g/100 g |
| Energy | 386 kcal/100 g |
| Sodium | 218 mg/100 g |
| Iron | 2.47 mg/100 g |
| Calcium | 843 mg/100 g |
| Magnesium | 53.0 mg/100 g |
| Thiamin (as thiamin hydrochloride) | 0.07 mg/100 g |
| Rivoflavin | 0.06 mg/100 g |
| Calciferol | not detected |
| | (LD 30 IU/100 g) |
| β-glucan | 0.4 g/100 g |
| Superoxide scavenging activity | 140 unit g |

### Test Example

Then, clinical test example of the healthy foods having antitumor/immunoactive ability described above of this invention is shown below.

### Test Example 1

Name of disease: Gliomatosis cerebri

Patient: Woman aged 36 at the time of crisis

Hospital: Ageo Chuo General Hospital/neurosergery section

Clinical Recording:

Jul. 19, 1995: Craniotomy extraction operation of tumor was applied. Then, chemotherapy of anticancer drugs was applied.

October, 1995: She could not drive a car because of the structure of visual field.

March, 1996: As a result of MRI inspection, recidivation of tumor was observed in the vicinity of the place where the tumor was extirpated. Since no effects of anticancer drugs were observed, she was notified that the reminder of her life would be one-half year. Treatments including chemotherapy such as of interferon and radiotherapy were applied but further treatments were postponed till the finding of new treatment method because they provide no therapeutical effects.

June 23, 1996: Therefore, the patient orally intook the healthy foods containing fermented crop products of this invention shown in the examples described above (hereinafter referred to as healthy foods of this invention) by from 10 g to 15 g per one day.

After 4 days from the start of administration, the progressing stricture of the visual field was stopped, and the visual field was started to recover.

December, 1996: As a comparison of the MRI inspection with that of the last time, no change was observed in the size of the tumor, and it was found not to be grown. The amount of the healthy foods of this invention to be administrated was reduced to one-half for examining the progress of the tumor.

March, 1997: It was confirmed by an MRI inspection that the recidivated tumor was completely annihilated, and the administration of the healthy foods of this invention was stopped from since to observe prognosis.

Sep. 22, 1998 : Shi was completely recovered by the finding of MRI inspection.

Jan. 19, 2000: No abnormality according to the finding of MRI inspection test.

Present state: The patient has not taken in any enzymatic foods of this invention since March 1997 up to now for four years and she leads daily life not different from those of healthy housewives of other families.

### Test Example 2

Name of disease: Multiplue Sclerosis

Patient: Woman aged 27 at the time of crisis.

Hospital: Tokyo University Hospital/neurointernal medicine section.

Clinical Recording:

January, 1998: Paralytic feeling began at first in hands and feet and the numbness was propagated to the whole body in October of the year. She tried inspections such as MRI inspection in the neurosurgery section of the Chiba Tokushu Kai Hospital but since the diagnosis could not be determined. She was examined again by the nuerointernal medicine section of the Hospital of Tokyo University. As a result, abnormalities were observed in her brain by MRI inspection for the head, and also in tibia by MRI inspection for legs, and she was diagnosed as suffered from multiple sclerosis and strongly recommended for admission to hospital. However, she did not enter the hospital since there were no therapeutic methods or medicines at present and, in addition, she could not be absent from her office for a long period of time.

October, 1998: She began to orally intake from 10 g to 20 g of thelhealthy foods of this invention per one day, and then the numbness of hands and feet and sense of exhaustion were eliminated since the middle of November, and a sign of recovery was observed.

Mar. 26, 1999: The abnormality of her brain disappeared as a result of MRI inspection in the Hospital of Tokyo University. In addition, it was analyzed that the shadow in the tibia was thinned. At this time, the amount of the enzymatic foods of this invention to be intaken was reduced to one half (10 g/day).

Dec. 10, 2000: As a result of the final diagnosis, all of the shadows in the brain and the tibia were eliminated completely, and she was recovered completely according to the result of MRI inspection in the hospital, so that there was no requirement for her to intake the healthy foods of this invention and she has not intake them at all at present.

Present state: The patient enjoys healthy daily life while working in her office with no worry.

Since the antitumor/immunoactive healthy foods of this invention comprise the mycelia of mushrooms mixed with the fermented crop products, it is considered that the polysaccharides (mainly β-glucanes) contained by a great mass to the mycelia of mushrooms gave damages to cancer cells, while cells in the plastic state recovered regeneration ability and were provided with immunocompetence by the fermented crop products, thereby reducing or annihilating the cancer cells.

The effects of the healthy foods of this invention is provided by the synergistic effects of the ingredients described above and, particularly, the regenerating ability of the cells is recovered since polysaccarides such as β-glucanes give damages to the cancer cells and ingesta such as carbonhydrates and proteins are supplemented to the cells under the effect of antioxidation.

In particular, the combination of mycelia of mushrooms with fermented crop products considerably improves the antitumor activity of mycelia of mushrooms. It is said that disorders of cells are caused by oxidation of cells which transform to aplastic cells, resulting in cancers. Mycelia of mushrooms have an antitumor activity but the activating performance for peripheral aplastic cells is poor. In such an aplastic state, the mycelia of mushrooms show only the transient effect and allow the cancer cells to grow. Accordingly, sufficient tumor-suppressing effect could not be obtained so far only by the mycelia of mushrooms.

The healthy food according to this invention prepared by mixing the mycelia of mushrooms and the fermented crop products exerts simultaneously on the cells (i) the antitumor activity by the polysaccharides, such as polysaccharides including β-glucans, for example, β-1-3 glucanase and/or β-1-4 glucanase, contained in the mycelia of mushrooms and (ii) immunocompetence activation effect, and the enhancing effect of cell regeneration, by the oxidation preventive enzymes and the nutritive ingredients contained in the fermented crop products. As a result, the antitumor effect is improved and the health enhancing effect as described in the test examples is obtained by the synergistic effects of them. That is, the healthy food according to the present invention has an advantageous feature capable of improving the tumor suppressing effect by exerting the antitumor activity of the mycelia of mushrooms while enhancing the regeneration function of the cells by the fermented crop products.

Since the mycelia of mushroom and the fermented crop products in the combination act on the cells simultaneously, they improve the regenerative function of living cells, by various kinds of oxidation preventive enzymes and nutritive ingredients, while damaging cancer cells by the anti-tumor activity of the mycelia of mushroom. For this reason, a long lasting anti-tumor effect and regenerative activity to the cells is obtained as shown in the Test Examples, which would not be possible with the use of mycelia of mushrooms alone.

For the fermented crop product, fermented products made of vegetable, fruits, root crops or mixtures thereof as disclosed in the Examples can be used. Mixtures of various crops are preferred, as they may contain various kinds of enzymes and nutritive ingredients, which are useful for promoting regeneration of cells. Fermentation is useful to improve cell activation and immunocompetence.

It is advantageous to reduce or suppress oxidation of cells and to remove oxides for regeneration and activation of the cells. Accordingly, the presence of an agent having a superoxide scavenging activity is useful to the other effects of the present invention. “Superoxide scavenging activity” means an activity of eliminating superoxides, and the unit of the superoxide scavenging activity corresponds to the activity of Enzyme Superoxide Dismutase (SOD) found by J. M. McCord and I. Fridovich. Thus, under this definition, 140 units/g as obtained in the Test Examples is a high value. The superoxide scavenging agent can be contained in the fermented crop products or added to the composition.

When calcium is added to the healthy foods described above, an antioxidation ability of cells can further be enhanced, to further improve the immunoactivity.

Various kinds of lytic enzymes are contained in the mycelia of mushrooms. Therefore, since polysaccharides, oxydoreductase and calcium as the components of the healthy foods are effectively intaken and absorbed into the cells, the antitumor components, and the antioxidation components act satisfactorily to improve the tumor suppressing effect.

What is claimed is

1. Healthy foods having antitumor/immunoactive activity comprising mycelia of mushrooms mixed with one or a plurality of fermented crop products.

2. Healthy foods having antitumor/immunoactive activity as defined in claim 1 comprising from 30 to 50% by weight of mycelia of mushrooms mixed with from 70 to 50% by weight of fermented crop products.

3. Healthy foods having antitumor/immunoactive activity comprising mycelia of mushrooms mixed with one or plurality of fermented crop products and calcium.

4. Healthy foods having antitumor/immunoactive activity as defined in claim 3 comprising from 30 to 50% by weight of mycelia of mushrooms, from 65 to 45% by weight of fermented crop products and from 0.5 to 3% by weight of calcium.

5. Healthy foods having antitumor/immunoactive activity as defined in claim 1, wherein the fermented crop products are a product prepared by adding yeast, water and maltose to one or a mixture of vegetables, fruits and crops and fermenting and ripen them.

6. Healthy foods having antitumor/immunoactive activity as defined in claim 5, wherein the fermented crop products are obtained by fermenting a mixture of broccoli, lettuce, apples, carrots and beets.

7. Healthy foods having antitumor/immunoactive activity as defined in claim 1, comprising an agent having oxide scavenging activity.

8. Healthy foods having antitumor/immunoactive activity as defined in claim 7, wherein the agent having oxide scavenging activity is contained in the fermented crop products.
